(19) United States
(12) Patent Application Publication
(10) Pub. No.: US 2010/0331751 A1
(43) Pub. Date: Dec. 30, 2010

(54) Burn Glove and Sock

(76) Inventors: E. Aubrey Woodroof, Carlsbad, CA (US); Amy Moss, Carlsbad, CA (US); Victoria Vandenberg, Carlsbad, CA (US); Karla Alvey, Carlsbad, CA (US)

Correspondence Address: STEVEN W. WEBB, 825 College Blvd., Suite 102620, Oceanside, CA, 92057, US

(21) Appl. No.: 12/493,238

(22) Filed: Jun. 28, 2009

(51) Int. Cl. A61F 13/00; A41D 19/00; A43B 17/00; D05B 23/00; A61M 35/00

(52) U.S. Cl. 602/43; 2/161.7; 2/239; 112/475.12; 112/475.09; 604/292

(57) ABSTRACT

An improved burn glove and sock is presented comprised of non-biological materials produced with a series of regularly-spaced pores and uniform three dimensional nylon structure/weave netting. Incorporated into the silicone/nylon weave structure are collagen peptides and other biological and non-biological agents selected for their therapeutic effect on burn wounds.

The gloves and sock come in left and right shapes, the gloves with opposable thumbs, an advance for the burn glove industry. The items are assembled from state-of-the-art skin substitute material in use for burn and wound dressings.

### FIELD OF THE INVENTION

This invention relates to the field of artificial skin substitutes used for wound and burn dressings, therapeutic burn dressings, burn gloves and socks.

### BACKGROUND OF THE INVENTION

A number of burn gloves exist in the current market that address the burn dressing problem for hands. There is currently no perfect product and there are still gaps in the capabilities of state-of-the-art burn gloves; however, the state-of-the-art Biobrane glove is superior to other burn gloves because of its excellent elongation in both the x and y direction. Biobrane does not contain a uniform three dimensional structure; it has voids at the pore sites. The porosity of Biobrane is about 1.2%; the present invention has a porosity of about 6%.

The present invention provides a precision, porous, stretchable membrane with uniform three dimensional structure over the entire glove surface in the shape of the human hand, with an opposable thumb, that fosters an ideal environment for the protection of existing cells and growth of new skin cells for the hands. The invention is also available in a sock shape, with separate toes. The present invention promotes rapid adhesion to the wound and other therapeutic goals, not offered by the state-of-the-art.

The present invention is made stretchable and tear resistant by using a fine knit nylon fabric and using a special, locking stretch stitch, designated #8 Size 2.0. Additionally, the porosity of the overlying membrane is greater in terms of placement and area of pores, compared to the state-of-the-art. The membrane is semi-permeable, allowing water vapor transmission and preventing fluid accumulation in the wound. Prefabricated shapes (gloves, socks, etc.) benefit the patient and surgeon by greatly reducing the Operating Room time to close these complex wounds. This, of course, reduces the cost for the procedure as well as the risk for the patient—less time under general anesthetic.

As a consequence, the porosity of the membrane comprising the burn glove and sock enhances the healing process, making it faster, safer and a painless experience. The membrane is also made capable of holding one or more biological agents that further increase the healing abilities of the covered area. These agents include but are not limited to biological agents such as collagen peptide, chondroitin 4 & 6 sulfate, Immuno-10 (or a similar purified fraction from the plant Aloe vera), vitamin C & E and others.

The present invention is easy to handle, flexible and stretchable, can be stored at room temperature, and is safe and sterile. Packaging and sterilizing of the invention is crucial. From a production and laboratory standpoint, manufacturing the invention is efficient in terms of time and materials. After manufacture, the product will be ready for immediate sterilization and shipping for use on patients.

### TECHNICAL BACKGROUND

Some ideal properties of a skin substitute from (Rusczak, 2006) and Properties from (Robert H. Demling):

a. Rapid and sustained adherence to wound surface and Inner surface structure that permits cell migration, proliferation and in growth of new tissue

### (The Most Important Criterion is Adherence)

b. Absence of antigenicity
c. Tissue compatible (Robert H. Demling)
d. Absence of local or systemic toxicity
e. Impermeable to exogenous microorganisms
f. Water vapor transmission similar to normal skin
g. Rapid and sustained adherence to wound surface
h. Conformal to surface irregularities
i. Elastic to permit motion of underlying tissue
j. Resistant to linear and shear stresses
k. Tensile strength to resist fragmentation (when removed)
l. Inhibition of wound surface flora and bacteria
m. Long shelf life, minimal storage requirements
n. Low cost
o. Minimize nursing care of wound
p. Minimize patient discomfort
q. Translucent properties to allow direct observation of healing
r. Reduce healing time
s. Patient acceptance

### PRIOR ART

### Tissue Based Skin Substitutes

Alloderm by LifeCell, Inc—AlloDerm is human tissue and is processed from donated human skin. The tissue goes through a cell removal process while retaining the important biochemical and structural components. AlloDerm is, thus, acellular human tissue. U.S. Pat. No. 6,933,326— Particulate acellular tissue matrix

Apligraf by Organogenesis Inc.—Apligraf is supplied as a living, bi-layered skin substitute: the epidermal layer is formed by human keratinocytes and has a well-differentiated stratum corneum; the dermal layer is composed of human fibroblasts in a bovine Type I collagen lattice. U.S. Pat. Nos. 4,485,096 5,106,949 5,536,656

Dermagraft by Smith & Nephew Inc—Dermagraft is a cryopreserved human fibroblast-derived dermal substitute; it is composed of fibroblasts, extra cellular matrix, and a bioabsorbable scaffold. U.S. Pat. No. 4,963,489: Three-dimensional cell and tissue culture system

Epicel by GenzymeBiosurgery—Epicel grafts are sheets of skin cells ranging from 2 to 8 cell layers thick. The grafts are grown or cultured from a postage stamp sized sample of patient's own healthy skin, which is sent to GenzymeBiosurgery for processing. The cells within the epidermis of the skin sample are separated and grown by a process called “tissue culture”, which involves feeding the cells with specific nutrients and maintaining strict climate controls so that the cells multiply to form sheets of skin. During this process, irradiated mouse cells, also referred to as 3T3 cells, and are used to promote cell growth and to ensure that there will be a sufficient number of grafts available as soon as possible for treatment. U.S. Pat. No. 6,964,869: Method and composition for skin grafts.

EZ Derm by Brennen Medical, Inc—A modified pigskin impregnated with a soluble silver compound intended for treatment of burns. Originally developed by Genetic Laboratories. US patent Numbers: Stabilized silver-ion amine complex compositions and methods U.S. Pat. No. 6,923,990. This is not a patent for EZ Derm but it is related to the silver that EZ-Derm uses

OrCel by Ortec International Inc.—A bilayered cellular matrix in which normal human allogeneic skin cells (epidermal keratinocytes and dermal fibroblasts) are cultured in two separate layers into a Type I bovine collagen sponge. Donor dermal fibroblasts are cultured on and within the porous sponge side of the collagen matrix while keratinocytes, from the same donor, are cultured on the coated, nonporous side of the collagen matrix.

TransCyte by Smith and Nephew, Inc—Consists of a polymer membrane and newborn human fibroblast cells cultured under aseptic conditions in vitro on a nylon weave. Prior to cell growth, this nylon weave is coated with porcine dermal collagen and bonded to a polymer membrane (silicone). This membrane provides a transparent synthetic epidermis when the product is applied to the burn.

As fibroblasts proliferate within the nylon weave during the manufacturing process, they secrete human dermal collagen, matrix proteins and growth factors. Following freezing, no cellular metabolic activity remains; however, the tissue matrix and bound growth factors are left intact. The human fibroblast-derived temporary skin substitute provides a temporary protective barrier. TransCyte is transparent and allows direct visual monitoring of the wound bed.

### Silver Based Skin Substitutes

### Aquacell Ag

### Silver Powered Antimicrobial Dressing

### ActiCoat

### Using Unique Silver Technology:

### SILCRYST Nanocrystalline

### Other Synthetic/Similar to Invention's Skin Substitute

Biobrane, Biobrane-L by Bertek Pharmaceuticals—Biobrane is a biocomposite temporary wound dressing constructed of an ultra thin, semipermeable silicone film with a nylon fabric partially imbedded into the film. The fabric presents to the wound bed a complex 3-D structure of trifilament thread to which porcine dermal collagen has been chemically bound. Blood/sera clot in the nylon matrix, thereby firmly adhering the dressing to the wound until epithelialization occurs. U.S. Pat. No. 4,725,279.

Integra Bilayer Matrix Wound Dressing by Integra LifeSciences Corp.—an advanced wound care device comprised of a porous matrix of cross-linked bovine tendon collagen and glycosaminoglycan and a semi-permeable polysiloxane (silicone) layer.

Laserskin by Fidia Advanced Biopolymers—Lam, P. K. et al; “Development and evaluation of a new composite Laserskin graft”, J of Trauma: Injury, Infection and Critical Care. 47, 1999. pp. 918-922.

Oasis Wound Matrix by Healthpoint—A biologically derived extra cellular matrix-based wound product that is compatible with human tissue. Unlike other collagen-based wound care materials, OASIS is unique because it is a complex scaffold that provides an optimal environment for a favorable host tissue response, a response characterized by restoration of tissue structure and function.

Glucan II—A smooth gas permeable polymeric layer attached to the mesh matrix. A highly advanced carbohydrate dressing with Beta-Glucan.

### SUMMARY OF THE INVENTION

The present invention uses technology described in U.S. patent application Ser. Nos. 12/049,321 and 12/348,412 to Woodroof and U.S. patent application Ser. No. 12/326,373 to Woodroof and Enright, all currently co-pending. This technology is for a porous, nylon mesh and silicone artificial skin substitute.

The burn glove invention is made of pieces cut from the skin substitute according to patterns sized for the human hand. The patterns support the creation of a glove for each hand, with opposable thumb. The pieces are sewn together with a mercerized cotton thread used for feminine hygiene products. The fingers of the glove and the toes of the sock are left open at the end to provide for hands with digits of any length. Biological/Active Component of the Skin Substitute

Additives—A silicone/nylon based membrane will adhere to a wound and protect it from the environment. The ideal environment it creates for the wound can be amplified through the addition of biological agents. Impregnation of the apparatus material itself with substances taken from the list of Vitamin E, Vitamin C, Chondroitin 4 Sulfate, Chondroitin 6 Sulfate and a highly purified fraction of aloe vera (Immuno-10™), and porcine collagen peptide improves the invention's therapeutic value.

The present invention is a porous, stretchable membrane with uniform three dimensional structure including the pore locations over the entire glove surface in the shape of the human hand, with an opposable thumb, that provides an ideal environment for the protection of existing cells and growth of new skin cells for the hands. The present invention promotes rapid adhesion to wounds, and other therapeutic goals, not offered by the state-of-the-art burn glove.

The present invention is made stretchable and tear resistant by using a fine knit nylon fabric. The porosity of the overlying membrane is greater in terms of placement and area of pores, compared to the state-of-the-art. The membrane is semi-permeable, allowing water vapor transmission and preventing fluid accumulation in the wound under the glove while providing a uniform three dimensional structure for optimal ingrowth of new skin cells: fibroblasts and keratinocytes.

This is the preferred embodiment of the invention. The technology to assemble the glove is listed as the preferred embodiment of this invention, and other methods are possible and are within the contemplation of this patent.

### BRIEF DESCRIPTION OF THE FIGURES

FIG. 1. Top View of Glove with a Human Hand Inserted

FIG. 2. Glove Patterns for the Right Hand

FIG. 3. Glove Patterns for the Left Hand

FIG. 4. Perspective view of Sock With Human Foot Inserted

FIG. 5A Sock Pattern Outer Foot

FIG. 5B Sock Pattern Upper Toes

FIG. 5C Sock Pattern Sole

FIG. 5D Sock Pattern Inner Foot

FIG. 5E Sock Patterns Lower Toes

### DETAILED SPECIFICATION

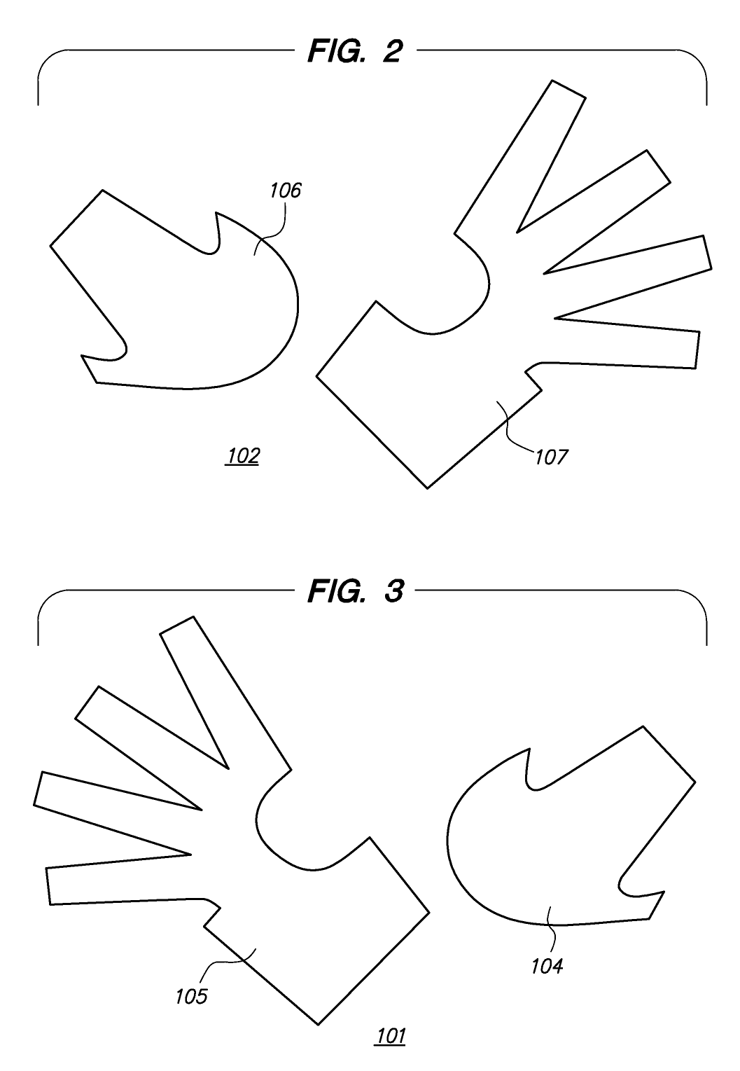

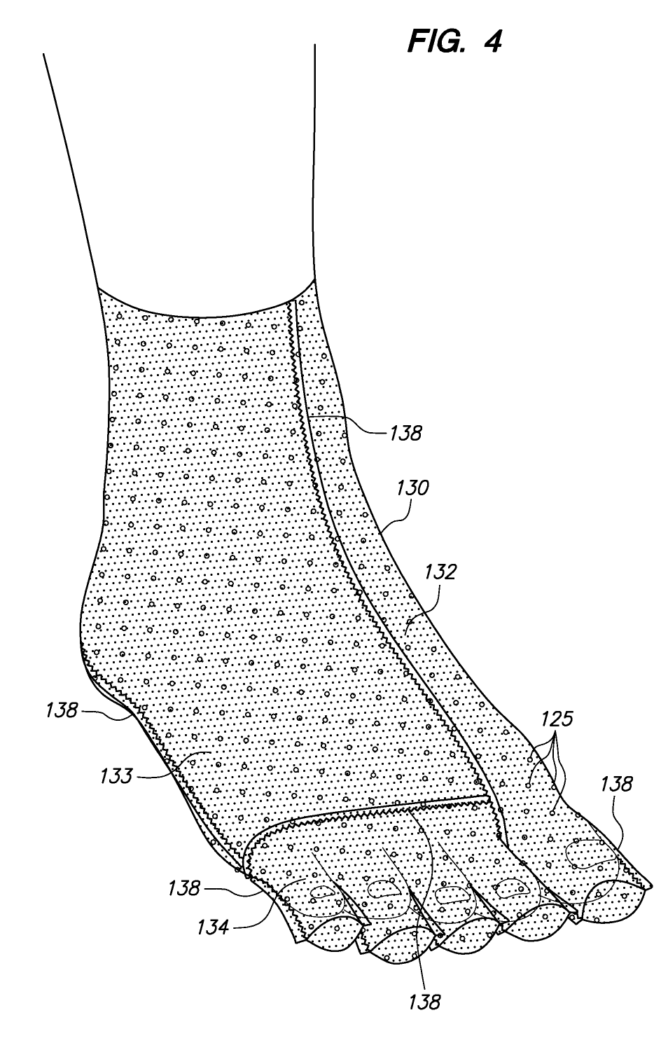

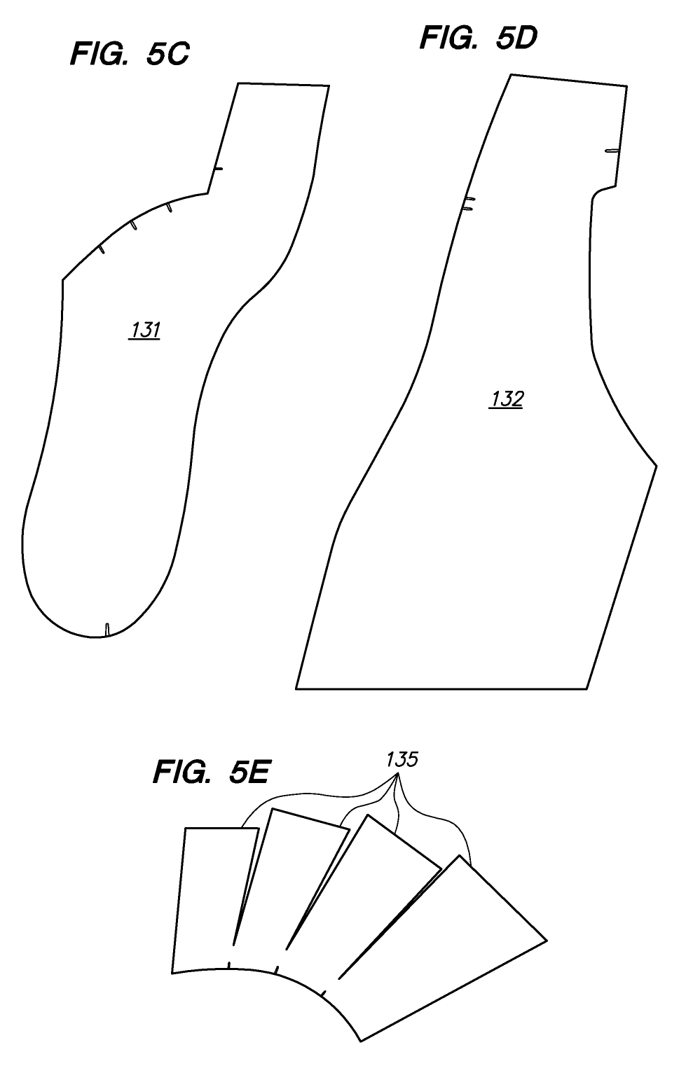

The invention 100 is comprised of several pieces of skin-substitute material 110, the invention assembled as either a left glove 101, right glove 102, a left sock 130, or a right sock. The patterns for the left glove are as shown in FIG. 3 and for the right glove in FIG. 2. The patterns for a left sock130 are shown in FIG. 5A through 5E.

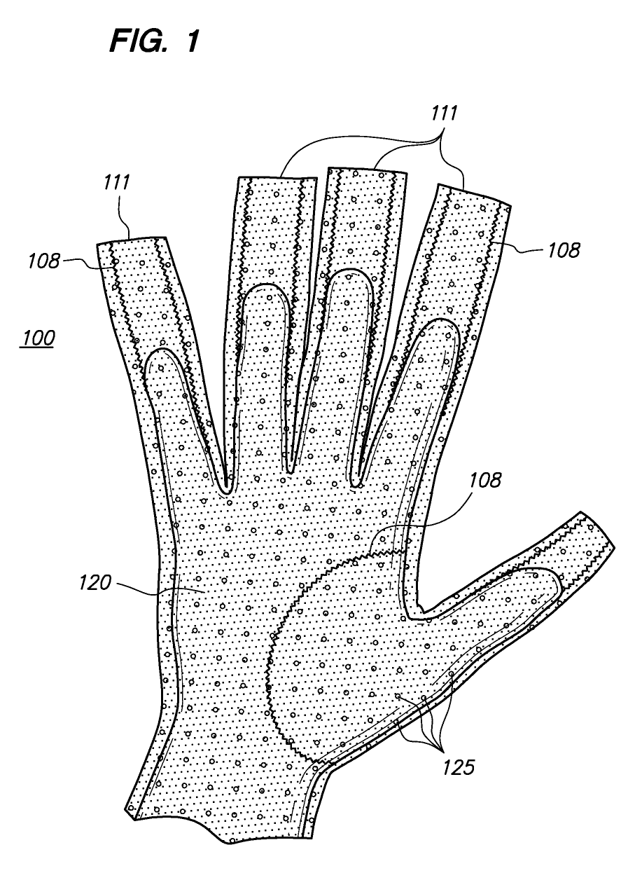

The left glove 101 is comprised of two subassemblies of material, the top subassembly and the bottom subassembly. Each subassembly is created by sewing a left thumb piece 104 to a left palm piece 105. The entire left glove 101 is made by sewing the two subassemblies together as shown in FIG. 1, with the nylon mesh layer of the skin-substitute material on the inside of the left glove 101.

Similarly, the right glove 102 is comprised of two sub assemblies formed from sewing a right thumb piece 106 to a right palm piece 107, the subassemblies sewn to each other with the mesh side of the skin-substitute material inside.

The sock, represented by a left sock130, is comprised of a sole 131, an inner foot 132, an outer foot 133, an upper toes 134, and an under toes 135 piece.

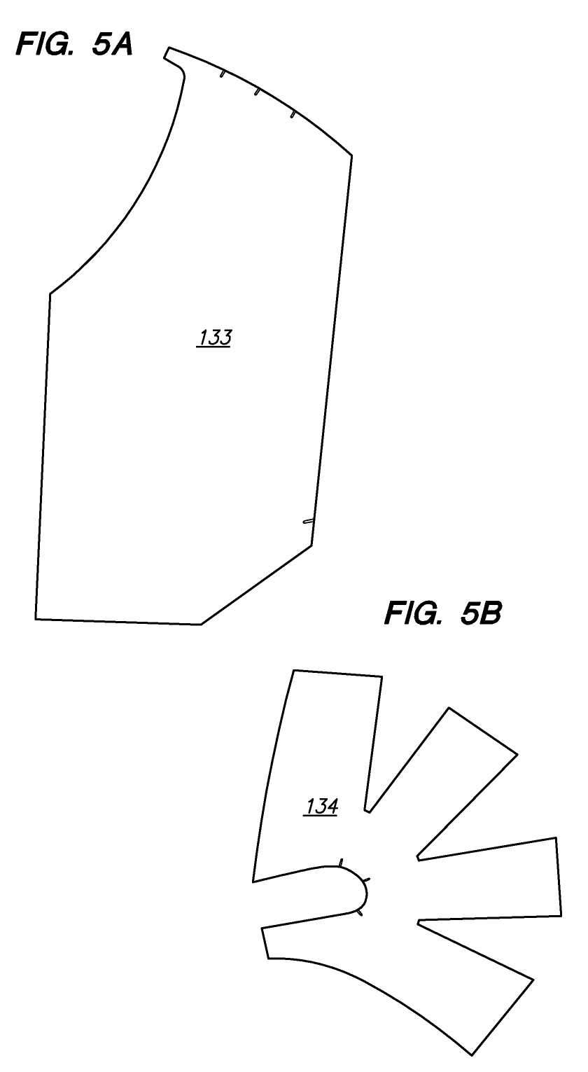

The seams 108 and 138, shown in FIG. 1 and FIG. 4, are sewn with thread in a stretchable stitch, in the preferred embodiment a mercerized cotton thread sewn in a locking stretch stitch. The patterns illustrated in FIG. 2, FIG. 3, and FIG. 5A through 5E can be made in a range of sizes from child's size to large adult. The fingers 111 of the gloves 100 and the toes 134, 135 of the sock 130 are left open at the end to accommodate fingers and toes of any length, and to improve air circulation.

The skin substitute, described in more detail in the above-referenced applications, is comprised of two layers. The membrane layer 120, on the outside of the glove 100 when assembled, possesses a plurality of pores 125 arranged across its surface in a regular pattern. The pores 125 are produced in its surface during manufacture by one of several means.

The nylon weave on the inside of the glove 100 when assembled is knitted and has a continuous, uniform three-dimensional structure. The uniform three-dimensional nature of the weave, when pressed against the skin of a burn wound, has improved therapeutic properties over existing, competing skin substitutes not available in the current art.

The nylon weave of the skin-substitute material can be impregnated with biogenic materials to deliver therapy to the wound site. These biogenic materials are selected from the list of Vitamin E, Vitamin C, Chondroitin 4 Sulfate, Chondroitin 6 Sulfate and a highly purified fraction of aloe vera (Immuno-10™), and porcine collagen peptide.

The apparatus and methods described are the preferred and alternate embodiments of this invention, but other methods are possible and are within the contemplation of this patent.

What is claimed is:

1. a burn glove system, the burn glove system comprised of a left glove, a right glove, and a sock,
the left glove comprised of two left glove subassemblies, each left glove subassembly comprised of a left thumb piece and a left palm piece held together with a connection means, the two left glove subassemblies held to each other with the connection means,
the right glove comprised of two right glove subassemblies, each right glove subassembly comprised of a right thumb piece and a right palm piece held together with the connection means, the two right glove subassemblies held to each other with the connection means,
the sock comprised of a sole piece, an inner foot piece, an under toe piece, an outer foot piece, and an upper toe piece,
the left glove and right glove and sock comprised of a skin substitute, the skin substitute formed of two layers, the inner layer a nylon mesh impregnated with biological and non-biological therapeutic agents, the outer layer a silicone sheath with perforations that is fixedly attached to the inner layer,
each glove and the sock assembled with the inner layer on the inner surface of the glove or sock and the outer layer on the outer surface of the glove.

2. A burn glove system as in claim 1, where the therapeutic agents are selected from the list comprised of Vitamin E, Vitamin C, Chondroitin 4 Sulfate, Chondroitin 6 Sulfate, a highly purified fraction of aloe vera (Immuno-10™ or similar purified fraction of Aloe vera), and porcine collagen peptide.

3. A burn glove system as in claim 1, where the connection means is sewing with mercerized cotton thread in a locking stretch stitch.

4. A method of assembly of a burn glove system as in claim 1 comprised of the steps of
obtaining flat sheets of skin substitute material,
cutting the sheets of skin substitute material into two left thumb, two left palm, two right thumb, and two right palm pieces using pre-developed size patterns,
using the connection means, sewing the left thumb pieces to the left palm pieces to form two left glove subassemblies, the right thumb pieces to the right palm pieces to form two right glove subassemblies,
using the connection means, sewing the two left glove subassemblies to each other to form a left glove, ensuring that the nylon mesh layer of the skin substitute is on the inside of the glove,
using the connection means, sewing the two right glove subassemblies to each other to form a right glove, ensuring that the nylon mesh layer of the skin substitute is on the inside of the glove.

5. A method of assembly of a burn glove system as in claim 1 comprised of the steps of
obtaining flat sheets of skin substitute material,
cutting the sheets of skin substitute material into inner foot, sole, under toes, upper toes, and outer foot pieces using pre-developed size patterns,
using the connection means, sewing the pieces into a sock, ensuring that the nylon mesh layer of the skin substitute is on the inside of the sock.
